(19) United States
(12) Patent Application Publication
(10) Pub. No.: US 2024/0373928 A1
(43) Pub. Date: Nov. 14, 2024

(54) ATOMIZATION CORE MODULE, ATOMIZER, AND ELECTRONIC ATOMIZATION APPARATUS

(71) Applicant: Jiangmen Smoore New Material Co., Ltd., Jiangmen (CN)

(72) Inventors: Shuai LUO, Jiangmen (CN); Zhiwen CHENG, Jiangmen (CN); Xiaoyong WANG, Jiangmen (CN)

(21) Appl. No.: 18/780,119

(22) Filed: Jul. 22, 2024

Related U.S. Application Data
(63) Continuation of application No. PCT/CN2022/142131, filed on Dec. 26, 2022.

(30) Foreign Application Priority Data
Jan. 23, 2022 (CN) 202210076046.6

(51) Int. Cl. A24F 40/46; A24F 40/10; A24F 40/48; A61M 11/04; A61M 15/06

(52) U.S. Cl. CPC A24F 40/46; A24F 40/10; A24F 40/48; A61M 11/042; A61M 15/06

(57) ABSTRACT

An atomization core module includes: a heating assembly having a heating unit, and a first electrode and a second electrode that are connected to the heating unit; a first connecting member having a mounting cavity, the heating assembly being disposed in the mounting cavity, the first electrode being electrically connected to the first connecting member; a second connecting member sleeved on an outer side of the first connecting member, the second electrode being electrically connected to the second connecting member; and an insulation member disposed between the first connecting member and the second connecting member so as to insulate the first connecting member from the second connecting member.

### CROSS-REFERENCE TO PRIOR APPLICATION

This application is a continuation of International Patent Application No. PCT/CN2022/142131, filed on Dec. 26, 2022, which claims priority to Chinese Patent Application No. 202210076046.6, filed on Jan. 23, 2022. The entire disclosure of both applications is hereby incorporated by reference herein.

### FIELD

This application relates to the field of electronic atomization technologies, and in particular, to an atomization core module, an atomizer, and an electronic atomization apparatus.

### BACKGROUND

An electronic atomization apparatus generally includes an atomizer and a main unit. The atomizer is configured to store and atomize an aerosol-generating substrate, and the main unit is configured to supply power to the atomizer and control the atomizer to atomize the aerosol-generating substrate.

In an existing electronic atomization apparatus, the atomizer is generally electrically connected to the main unit by using an ejector pin or an elastic pin. This structure for implementing the electrical connection has a complex design, and has high assembly difficulty.

### SUMMARY

In an embodiment, the present invention provides an atomization core module, comprising: a heating assembly comprising a heating unit, and a first electrode and a second electrode that are connected to the heating unit; a first connecting member having a mounting cavity, the heating assembly being disposed in the mounting cavity, the first electrode being electrically connected to the first connecting member; a second connecting member sleeved on an outer side of the first connecting member, the second electrode being electrically connected to the second connecting member; and an insulation member disposed between the first connecting member and the second connecting member so as to insulate the first connecting member from the second connecting member.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic diagram of a structure of an electronic atomization apparatus according to an embodiment of this application;

FIG. 2 is a schematic diagram of an exploded structure of an atomizer in the electronic atomization apparatus provided in FIG. 1;

FIG. 3 is a schematic diagram of a cross-sectional structure of the atomizer in the electronic atomization apparatus provided in FIG. 1;

FIG. 4 is a schematic diagram of an exploded structure of an atomization core module in the atomizer provided in FIG. 2;

FIG. 5 is a schematic diagram of a cross-sectional structure in a first direction of the atomization core module provided in FIG. 4;

FIG. 6 is a schematic diagram of a cross-sectional structure in a second direction of the atomization core module provided in FIG. 4;

FIG. 7 is a schematic diagram of a structure of a first connecting member in the atomization core module provided in FIG. 4;

FIG. 8 is a schematic diagram of a structure of a second connecting member in the atomization core module provided in FIG. 4;

FIG. 9 is a schematic diagram of an exploded structure of a heating assembly in the atomization core module provided in FIG. 4;

FIG. 10 is a schematic diagram of a structure from another angle of a heating unit in the heating assembly provided in FIG. 9; and

FIG. 11 is a schematic diagram of a structure from another angle of a second seal member in the heating assembly provided in FIG. 9.

### DETAILED DESCRIPTION

In an embodiment, the present invention provides an atomization core module, an atomizer, and an electronic atomization apparatus provided in this application resolve a technical problem that a structure for electrically connecting an atomizer and a main unit in an electronic atomization apparatus in the related art is complex.

In an embodiment, the present invention provides an atomization core module, including a heating assembly, a first connecting member, a second connecting member, and an insulation member; the heating assembly includes a heating unit, and a first electrode and a second electrode that are connected to the heating unit; the first connecting member has a mounting cavity; the heating assembly is disposed in the mounting cavity; the first electrode is electrically connected to the first connecting member; the second connecting member is sleeved on an outer side of the first connecting member; the second electrode is electrically connected to the second connecting member; and the insulation member is disposed between the first connecting member and the second connecting member, to insulate the first connecting member from the second connecting member.

The second connecting member includes a connection column, the first connecting member is provided with a communication hole, and the connection column penetrates the communication hole and is electrically connected to the second electrode.

The first connecting member includes a first body portion, a surface of an end of the first body portion is provided with a mounting groove, and the mounting groove forms the mounting cavity; and an atomization surface of the heating unit is disposed toward a bottom surface of the mounting groove.

The first electrode is in contact with the bottom surface of the mounting groove, to electrically connect the first electrode to the first connecting member.

A bottom wall of the mounting groove is provided with a first groove, an inner surface of the first groove has a protrusion, and the protrusion extends in a depth direction of the first groove; and an end surface of the protrusion that is close to the mounting groove is electrically connected to the first electrode.

A bottom wall of the mounting groove is provided with a first groove, and an atomization cavity is formed between the atomization surface of the heating unit and an inner surface of the first groove.

The first connecting member further includes a first extending portion connected to the first body portion, the first extending portion is provided with a first through hole in communication with the first groove, and the first through hole is configured to communicate the atomization cavity with outside air.

An outer diameter of the first extending portion is less than an outer diameter of the first body portion; and the bottom wall of the first groove is provided with the communication hole.

The second connecting member further includes a second body portion and a second extending portion that are connected to each other; a surface of an end of the second body portion is provided with a second groove, and the first body portion is disposed in the second groove; the second extending portion is provided with a second through hole in communication with the second groove, and the first extending portion is disposed in the second through hole; and the connection column is disposed on a bottom surface of the second groove.

An outer diameter of the second extending portion is less than an outer diameter of the second body portion.

An outer surface of the second extending portion is provided with a thread, to connect the atomization core module to a main unit.

The insulation member includes a hollow insulation tube and an annular flange; the hollow insulation tube is disposed between the first extending portion and the second extending portion; and the annular flange is connected to an outer surface of an end of the hollow insulation tube and disposed between the first body portion and a bottom wall of the second groove.

The heating assembly further includes a seal member, and the seal member is configured to seal a periphery of the heating unit; the heating unit includes a porous liquid guide member and a heating element; and the porous liquid guide member includes a liquid absorbing surface and an atomization surface, and the heating element is disposed on the atomization surface.

To resolve the foregoing technical problem, a second technical solution provided in this application is as follows. An atomizer is provided, including an atomization tube, an atomization core module, and a suction nozzle component; the atomization tube includes a first end and a second end that are opposite to each other; the atomization core module is the atomization core module according to any one of the above; the atomization core module is disposed at the first end of the atomization tube and plugs the first end of the atomization tube; the suction nozzle component is disposed at the second end of the atomization tube; the suction nozzle component forms a first channel; the atomization tube, the atomization core module, and the suction nozzle component cooperate to form a liquid storage cavity, and the liquid storage cavity is configured to store an aerosol-generating substrate; a heating unit of the atomization core module is configured to atomize the aerosol-generating substrate to generate an aerosol; and the first channel is configured to output the aerosol.

A first connecting member includes a first body portion, a surface of an end of the first body portion is provided with a mounting groove, and the mounting groove forms a mounting cavity; a bottom wall of the mounting groove is provided with a first groove, and an atomization cavity is formed between an atomization surface of the heating unit and an inner surface of the first groove; and the atomization tube forms a second channel, and the second channel communicates an atomization cavity with the first channel.

An end portion of a heating assembly that is close to the suction nozzle component is provided with a liquid outlet, and the liquid outlet enables the liquid storage cavity to be in fluid communication with the heating assembly.

The heating assembly further includes a seal member, and the seal member is configured to seal a periphery of the heating unit; the seal member is provided with at least one notch, and the notch forms the liquid outlet.

To resolve the foregoing technical problem, a third technical solution provided in this application is as follows. An electronic atomization apparatus is provided, including an atomizer and a main unit; the atomizer is configured to store and atomize an aerosol-generating substrate; the atomizer is the atomizer according to any one of the above; and the main unit is configured to supply power to the atomizer and control the atomizer to atomize the aerosol-generating substrate.

Different from the related art, according to the atomization core module, the atomizer, and the electronic atomization apparatus provided in this application, the atomization core module includes a heating assembly, a first connecting member, a second connecting member, and an insulation member; the heating assembly includes a heating unit, and a first electrode and a second electrode that are connected to the heating unit; the first connecting member has a mounting cavity; the heating assembly is disposed in the mounting cavity; the first electrode is electrically connected to the first connecting member; the second connecting member is sleeved on an outer side of the first connecting member; the second electrode is electrically connected to the second connecting member; and the insulation member is disposed between the first connecting member and the second connecting member, to insulate the first connecting member from the second connecting member. Through the foregoing arrangement, a quantity of elements for electrically connecting the heating assembly to the main unit is reduced, and the assembly difficulty is reduced.

The technical solutions in embodiments of this application are clearly and completely described below with reference to the accompanying drawings in the embodiments of this application. It is clear that the described embodiments are merely some rather than all of the embodiments of this application. All other embodiments obtained by a person of ordinary skill in the art based on the embodiments of this application without creative efforts shall fall within the protection scope of this application.

In the following descriptions, for the purpose of illustration rather than limitation, specific details such as a specific system structure, an interface, and a technology are proposed to thoroughly understand this application.

The terms “first”, “second”, and “third” in this application are merely intended for a purpose of description, and shall not be understood as indicating or implying relative significance or implicitly indicating a quantity of indicated technical features. Therefore, features defining “first”, “second”, and “third” can explicitly or implicitly include at least one of the features. In the descriptions of this application, “a plurality of” means at least two, such as two and three unless it is specifically defined otherwise. All directional indications (for example, upper, lower, left, right, front, and back) in the embodiments of this application are only used for explaining relative position relationships, movement situations, or the like among the various components in a specific posture (as shown in the accompanying drawings). If the specific posture changes, the directional indications change accordingly. In the embodiments of this application, the terms “include”, “have”, and any variant thereof are intended to cover a non-exclusive inclusion. For example, a process, method, system, product, or device that includes a series of steps or units is not limited to the listed steps or units, but further optionally includes a step or unit that is not listed, or further optionally includes another step or component that is intrinsic to the process, method, product, or device.

“Embodiment” mentioned in this specification means that particular features, structures, or characteristics described with reference to the embodiment may be included in at least one embodiment of this application. The term appearing at different positions of this specification may not refer to the same embodiment or an independent or alternative embodiment that is mutually exclusive with another embodiment. A person skilled in the art explicitly or implicitly understands that the embodiments described in this specification may be combined with other embodiments.

This application is further described in detail below with reference to the accompanying drawings and embodiments.

Referring to FIG. 1, FIG. 1 is a schematic diagram of a structure of an electronic atomization apparatus according to an embodiment of this application. This embodiment of this application provides an electronic atomization apparatus 100. The electronic atomization apparatus 100 may be configured to atomize an aerosol-generating substrate. The electronic atomization apparatus 100 includes an atomizer 1 and a main unit 2 that are electrically connected to each other.

The atomizer 1 is configured to store the aerosol-generating substrate and atomize the aerosol-generating substrate, to generate an aerosol that can be inhaled by a user. The atomizer 1 specifically may be used in different fields, for example, medical treatment, cosmetics, and leisure inhaling. In a specific embodiment, the atomizer 1 may be used in an electronic aerosol atomization apparatus, and configured to atomize an aerosol-generating substrate and generate an aerosol for a smoker to inhale. In all the following embodiments, leisure inhaling is used as an example. Certainly, in another embodiment, the atomizer 1 may alternatively be used in a hair spray device to atomize hair spray for hair styling; or a device for treating diseases of the upper and lower respiratory system, to atomize medical drugs.

For a specific structure and function of the atomizer 1, reference may be made to a specific structure and function of the atomizer 1 in any of the following embodiments, and a same or similar technical effect can be achieved. Details are not described herein.

The main unit 2 includes a battery and a controller. The battery is configured to supply power to operation of the atomizer 1, so that the atomizer 1 can atomize an aerosol-generating substrate to form an aerosol. The controller is configured to control the atomizer 1 to atomize the aerosol-generating substrate. The main unit 2 further includes other elements such as a battery holder and an airflow sensor.

The atomizer 1 and the main unit 2 may be integrally arranged or may be detachably connected to each other, which may be designed according to a specific requirement.

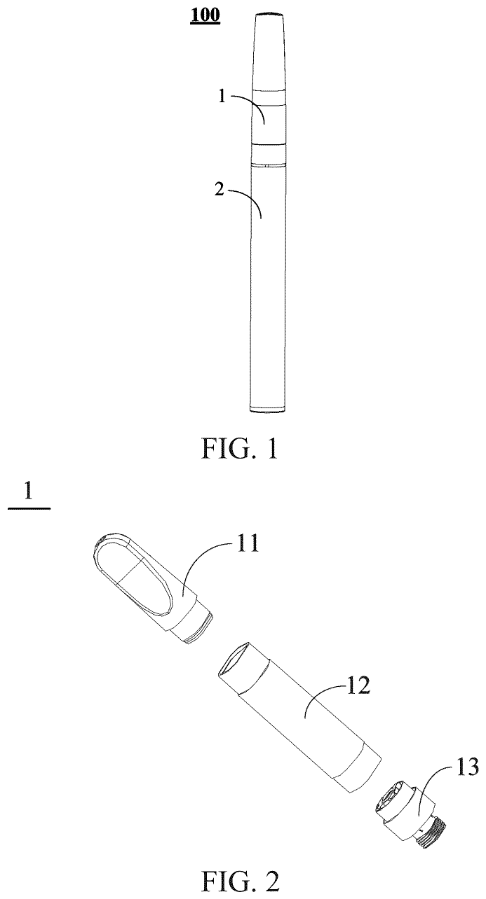

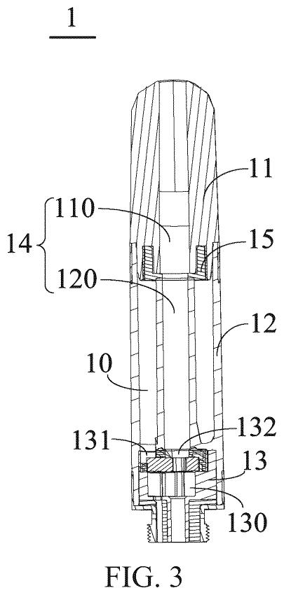

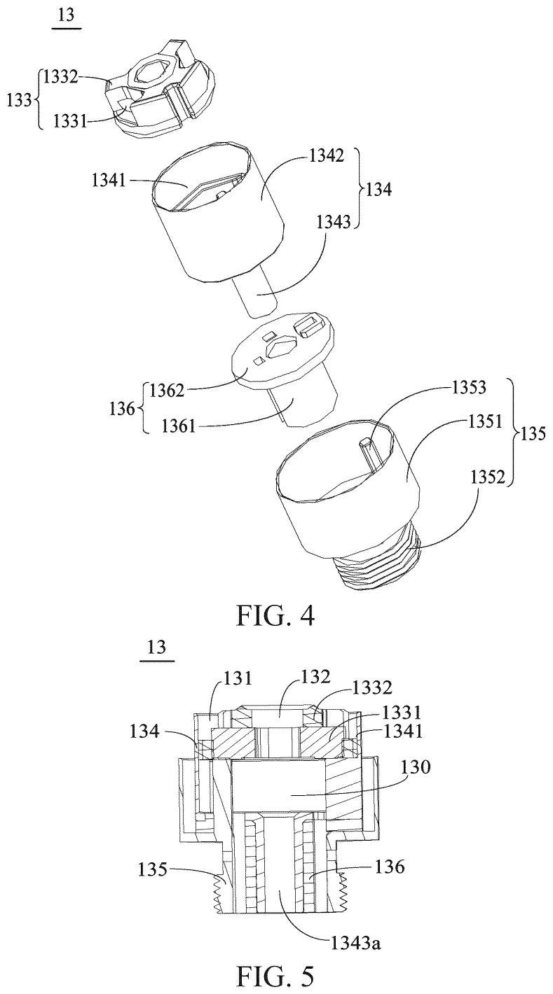

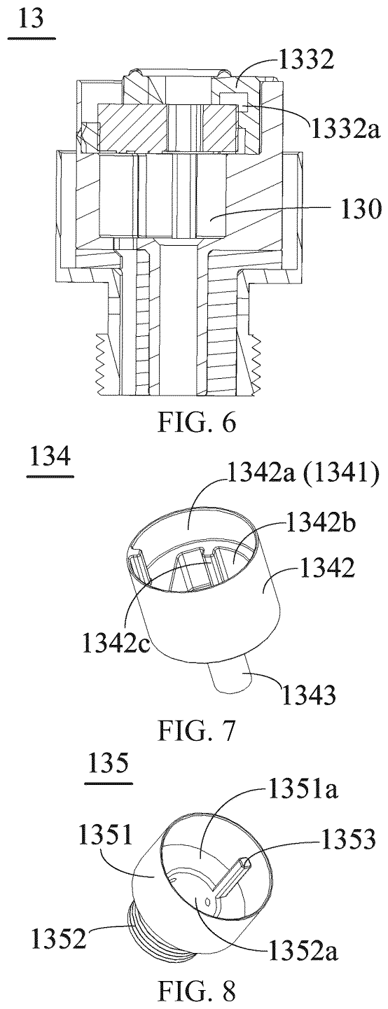

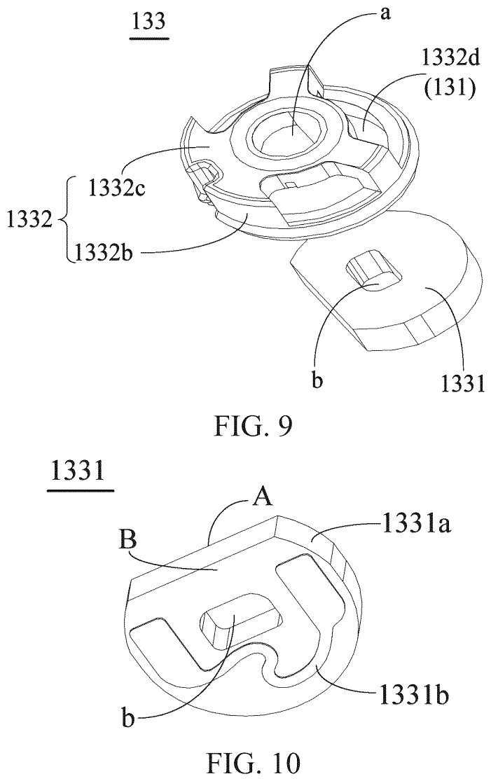

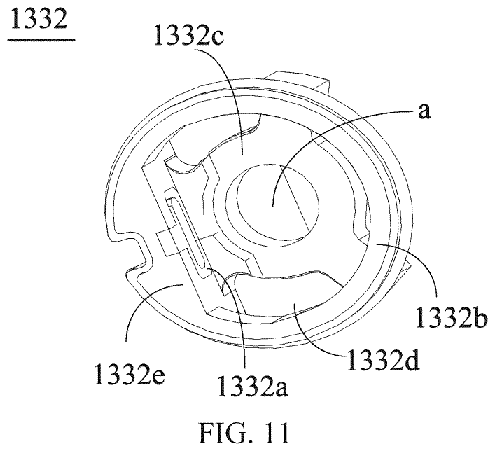

Referring to FIG. 2 to FIG. 8, FIG. 2 is a schematic diagram of an exploded structure of an atomizer in the electronic atomization apparatus provided in FIG. 1, FIG. 3 is a schematic diagram of a cross-sectional structure of the atomizer in the electronic atomization apparatus provided in FIG. 1, FIG. 4 is a schematic diagram of an exploded structure of an atomization core module in the atomizer provided in FIG. 2, FIG. 5 is a schematic diagram of a cross-sectional structure in a first direction of the atomization core module provided in FIG. 4, FIG. 6 is a schematic diagram of a cross-sectional structure in a second direction of the atomization core module provided in FIG. 4, FIG. 7 is a schematic diagram of a structure of a first connecting member in the atomization core module provided in FIG. 4, FIG. 8 is a schematic diagram of a structure of a second connecting member in the atomization core module provided in FIG. 4, FIG. 9 is a schematic diagram of an exploded structure of a heating assembly in the atomization core module provided in FIG. 4, FIG. 10 is a schematic diagram of a structure from another angle of a heating unit in the heating assembly provided in FIG. 9, and FIG. 11 is a schematic diagram of a structure from another angle of a second seal member in the heating assembly provided in FIG. 9.

Referring to FIG. 2 and FIG. 3, the atomizer 1 includes a suction nozzle component 11, an atomization tube 12, and an atomization core module 13. The atomization tube 12 includes a first end and a second end that are opposite to each other. The atomization core module 13 is disposed at the first end of the atomization tube 12 and plugs the first end of the atomization tube 12. Specifically, the atomization core module 13 is partially disposed inside the atomization tube 12, and partially disposed outside the atomization tube 12. The suction nozzle component 11 is disposed at the second end of the atomization tube 12. An assembly process of the atomizer 1 is as follows. First, the atomization core module 13 is in interference fit with the first end of the atomization tube 12, then the aerosol-generating substrate is injected from the second end of the atomization tube 12 into inner space of the atomization tube 12, and then the suction nozzle component 11 is pressed to the second end of the atomization tube 12 through riveting. In other words, the suction nozzle component 11, the atomization tube 12, and the atomization core module 13 in this embodiment may be separately assembled, and then assembled together to form the atomizer 1.

The suction nozzle component 11, the atomization tube 12, and the atomization core module 13 cooperate to form a liquid storage cavity 10. The liquid storage cavity 10 is configured to store the aerosol-generating substrate. An end portion of the atomization core module 13 that is close to the suction nozzle component 11 is provided with a liquid outlet 131. The liquid outlet 131 enables the liquid storage cavity 10 to be in fluid communication with the atomization core module 13, so that the aerosol-generating substrate in the liquid storage cavity 10 enters the atomization core module 13. The atomization core module 13 is configured to atomize the aerosol-generating substrate, to generate an aerosol through heating.

A first channel 110 is formed in the suction nozzle component 11, and a second channel 120 is formed in the atomization tube 12. The first channel 110 is in communication with the second channel 120 to form an air outlet channel 14. An end portion of the atomization core module 13 that is close to the atomization tube 12 is provided with a vapor outlet 132. The vapor outlet 132 is in communication with the air outlet channel 14, and the aerosol generated by the atomization core module 13 by atomizing the aerosol-generating substrate enters the air outlet channel 14 through the vapor outlet 132, to be inhaled by a user. In other words, the first channel 110 and the second channel 120 are configured to output the aerosol. A first seal member 15 is provided between the suction nozzle component 11 and the atomization tube 12, to seal a connection part between the first channel 110 and the second channel 120, to prevent the aerosol from leaking from the connection part between the first channel 110 and the second channel 120. Referring to FIG. 4 and FIG. 5, the atomization core module 13 includes a heating assembly 133, a first connecting member 134, a second connecting member 135, and an insulation member 136. The heating assembly 133 includes a heating unit 1331, and a first electrode and a second electrode that are connected to the heating unit 1331. The heating unit 1331 is configured to atomize the aerosol-generating substrate. The first connecting member 134 has a mounting cavity 1341. The heating assembly 133 is disposed in the mounting cavity 1341. The first electrode of the heating assembly 133 is electrically connected to the first connecting member 134. An end portion of the first connecting member 134 that is away from the heating assembly 133 is configured to be electrically connected to the main unit 2. The second connecting member 135 is sleeved on an outer side of the first connecting member 134; and the second electrode of the heating assembly 133 is electrically connected to the second connecting member 135, and an end portion of the second connecting member 135 that is away from the heating assembly 133 is configured to be electrically connected to the main unit 2. The insulation member 136 is sleeved between the first connecting member 134 and the second connecting member 135 to insulate the first connecting member 134 from the second connecting member 135. Optionally, materials of the first connecting member 134 and the second connecting member 135 are metal, provided that the materials are electrically conductive; and a material of the insulation member 136 is plastic, provided that the material is insulated.

An assembly process of the atomization core module 13 may be as follows. The insulation member 136 is first pressed onto the first connecting member 134, then the insulation member 136 is pressed onto the second connecting member together with the first connecting member 134, and then the heating assembly 133 is mounted to the first connecting member, to form the atomization core module 13.

The heating unit 1331 is electrically connected to the main unit 2 through the first electrode, the second electrode, the first connecting member 134, and the second connecting member 135. The first connecting member 134 and the second connecting member 135 not only serve as a conducting member to electrically connect the heating unit 1331 to the main unit 2, but also serve as a structural member for supporting and fixing the heating assembly 133. In comparison with an existing atomizer in which the heating unit is electrically connected to the main unit through an ejector pin or an elastic pin, a quantity of elements is reduced and assembly difficulty is reduced. In addition, the heating assembly 133, the first connecting member 134, the second connecting member 135, and the insulation member 136 are modularized, so that an overall assembly structure of the electronic atomization apparatus is simple, to help improve product stability.

Referring to FIG. 5, the heating assembly 133 is provided with a through hole at a position corresponding to the second channel 120. The through hole forms the vapor outlet 132, so that the aerosol generated through atomization by the heating assembly 133 enters the second channel 120 through the vapor outlet 132. An end portion of the heating assembly 133 that is close to the suction nozzle component 11 is provided with a liquid outlet 131. The liquid outlet 131 enables the liquid storage cavity to be 10 in fluid communication with the heating assembly 133.

Specifically, referring to FIG. 9 to FIG. 11, the heating assembly 133 further includes a second seal member 1332. The second seal member 1332 seals a periphery of the heating unit 1331. The second seal member 1332 is provided with a liquid outlet 131, so that the aerosol-generating substrate enters the heating unit 1331.

The second seal member 1332 includes an annular side wall 1332b and a top wall 1332c that are connected to each other. The heating unit 1331 is disposed in an inner space surrounded and formed by the second seal member 1332. Optionally, the heating unit 1331 is in interference fit with the second seal member 1332. A shape and a size of the heating unit 1331 are set in cooperation with a shape and a size of the second seal member 1332. The second seal member 1332 is provided with at least one notch 1332d, and the notch 1332d extends from the top wall 1332c to the annular side wall 1332b, so that when the heating unit 1331 is disposed in the inner space formed by the second seal member 1332, the heating unit 1331 is partially exposed. Therefore, the aerosol-generating substrate is in fluid communication with the heating unit 1331. It may be understood that the notch 1332d forms the liquid outlet 131, and the aerosol-generating substrate enters the heating unit 1331 through the liquid outlet 131. In this embodiment, the annular side wall 1332b is a ring, and the top wall 1332c is a disc.

The top wall 1332c is provided with a through hole a. Optionally, the through hole a is located at a central position of the top wall 1332c. The heating unit 1331 is provided with a through hole b. The through hole a is disposed corresponding to the through hole b. The through hole a and the through hole b cooperate to form the vapor outlet 132. An inner surface of the annular side wall 1332b is provided with a bump 1332e, and the bump 1332e is provided with a vent groove 1332a. With reference to FIG. 6, the vent groove 1332a is in communication with the liquid storage cavity 10 and the atomization cavity 130, to exchange air for the liquid storage cavity 10, so that sufficient liquid supply to the heating assembly 133 is ensured and a dry heating phenomenon is avoided.

The heating unit 1331 includes a porous liquid guide member 1331a and a heating clement 1331b. The heating element 1331b may be a heating film, a metal mesh, a metal sheet, or the like. The porous liquid guide member 1331a includes a liquid absorbing surface A and an atomization surface B. The heating element 1331b is disposed on the atomization surface B of the porous liquid guide member 1331a. The porous liquid guide member 1331a guides the aerosol-generating substrate to the atomization surface B by using a capillarity force of the aerosol-generating substrate, and an aerosol is generated by the heating element 1331b. The heating unit 1331 is a high thermal conductivity heating unit. In another embodiment, for the heating unit 1331, a conductive and porous liquid guide member, for example, a porous conductive ceramic, may be used. Because the porous conductive ceramic can both guide liquid and electrically heat, no heating element needs to be specifically disposed.

Referring to FIG. 4, FIG. 5, and FIG. 7, the first connecting member 134 includes a first body portion 1342 and a first extending portion 1343 connected to the first body portion 1342. Optionally, the first body portion 1342 and the first extending portion 1343 are integrally formed. A surface of an end of the first body portion 1342 is provided with a mounting groove 1342a, and the mounting groove 1342a forms the mounting cavity 1341. The atomization surface of the heating unit 1331 is disposed toward a bottom surface of the mounting groove 1342a, that is, the atomization surface of the heating unit 1331 faces downward. A bottom wall of the mounting groove 1342a is provided with a first groove 1342b. An atomization cavity 130 is formed between the atomization surface of the heating unit 1331 and an inner surface of the first groove 1342b, and an aerosol generated by the heating unit 1331 through atomization is released into the atomization cavity 130. The atomization cavity 130 is in communication with the air outlet channel 14 through the vapor outlet 132. The first extending portion 1343 is provided with a first through hole 1343a in communication with the first groove 1342b. The first through hole 1343a is configured to communicate the atomization cavity 130 with outside air. It may be understood that, the external air enters the atomization cavity 130 through the first through hole 1343a, and then flows into the air outlet channel 14 through the vapor outlet 132. In this embodiment, the first body portion 1342 and the first extending portion 1343 are both cylindrical, disposed coaxially, and integrally formed. A diameter of the first body portion 1342 is greater than a diameter of the first extending portion 1343.

In an implementation, the first electrode of the heating assembly 133 is in contact with the bottom surface of the mounting groove 1342a, so that the first electrode is electrically connected to the first connecting member 134.

In an implementation, the inner surface of the first groove 1342b includes a protrusion 1342c. The protrusion 1342c extends in a depth direction of the first groove 1342b (as shown in FIG. 7). An end surface of the protrusion 1342c that is close to the mounting groove 1342a is electrically connected to the first electrode. Optionally, a length of the protrusion 1342c is the same as a depth of the first groove 1342b. The protrusion 1342c and the first body portion 1342 are integrally formed.

Referring to FIG. 4 and FIG. 8, the second connecting member 135 includes a second body portion 1351 and a second extending portion 1352 that are connected to each other, and a connection column 1353. A surface of an end of the second body portion 1351 is provided with a second groove 1351a. The second extending portion 1352 is provided with a second through hole 1352a in communication with the second groove 1351a. The connection column 1353 is disposed on a bottom surface of the second groove 1351a, and the connection column 1353 is spaced from a port of the second through hole 1352a. The bottom wall of the first groove 1342b is provided with a communication hole, that is, the first connecting member 134 is provided with the communication hole.

The connection column 1353 penetrates the communication hole and is electrically connected to the second electrode of the heating assembly 133. Optionally, the second body portion 1351, the second extending portion 1352, and the connection column 1353 are integrally formed. In this embodiment, the second body portion 1351 and the second extending portion 1352 are both cylindrical and are disposed coaxially.

A diameter of the second body portion 1351 is greater than a diameter of the second extending portion 1352. The second groove 1351a and the second through hole 1352a are both cylindrical. The connection column 1353 is disposed on an edge of the bottom wall of the second groove 1351a that is close to the second through hole 1352a.

It may be understood that, the first electrode of the heating assembly 133 is electrically connected to the main unit 2 through the protrusion 1342c of the first connecting member 134, and a contact area between the first electrode and the protrusion 1342c is large. The second electrode of the heating assembly 133 is electrically connected to the main unit 2 through the connection column 1353 of the second connecting member 135, and a contact area between the second electrode and the connection column 1353 is large, to ensure the stability of electrical connection.

When the first connecting member 134 and the second connecting member 135 are sleeved, the first body portion 1342 of the first connecting member 134 is disposed in the second groove 1351a, and the first extending portion 1343 of the first connecting member 134 is disposed in the second through hole 1352a, so that the second connecting member 135 is sleeved on an outer side of the first connecting member 134.

The second extending portion 1352 is further configured to be connected to the main unit 2. Optionally, an outer surface of the second extending portion 1352 is provided with a thread to be connected to the main unit 2. To be specific, the atomization core module 13 may be detachably connected to the main unit 2 through the thread. It may be understood that, due to fixed connection between the suction nozzle component 11, the atomization tube 12, and the atomization core module 13, the atomization core module 13 is connected to the main unit 2 through the thread on the outer surface of the second extending portion 1352, in other words, the atomizer 1 is detachably connected to the main unit 2.

Optionally, shapes of the first body portion 1342, the first extending portion 1343, the second body portion 1351, and the second extending portion 1352 are all cylinders. An outer diameter of the first extending portion 1343 is less than an outer diameter of the first body portion 1342, and an outer diameter of the second extending portion 1352 is less than an outer diameter of the second body portion 1351, so that a shape the electronic atomization apparatus is flat after the atomization core module 13 is connected to the main unit 2.

Referring to FIG. 4, the insulation member 136 includes a hollow insulation tube 1361 and an annular flange 1362. The hollow insulation tube 1361 is disposed between the first extending portion 1343 and the second extending portion 1352. The annular flange 1362 is connected to an outer surface of an end portion of the hollow insulation tube 1361. The annular flange 1362 is disposed between the first body portion 1342 and the bottom wall of the second groove 1351a. It may be understood that, the insulation member 136 is cooperatively arranged with the first connecting member 134 and the second connecting member 135, provided that the first connecting member 134 can be insulated from the second connecting member 135. Further, the annular flange 1362 further includes a through hole for the connection column 1353 to penetrate.

With reference to FIG. 3, the first body portion 1342 of the first connecting member 134 is disposed inside the atomization tube 12, and the inner surface of the side wall of the second groove 1351a of the second connecting member 135 fits an outer surface of the atomization tube 12, to plug the second end of the atomization tube 12.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. An atomization core module, comprising:
a heating assembly comprising a heating unit, and a first electrode and a second electrode that are connected to the heating unit;
a first connecting member having a mounting cavity, the heating assembly being disposed in the mounting cavity, the first electrode being electrically connected to the first connecting member;
a second connecting member sleeved on an outer side of the first connecting member, the second electrode being electrically connected to the second connecting member; and
an insulation member disposed between the first connecting member and the second connecting member so as to insulate the first connecting member from the second connecting member.

2. The atomization core module of claim 1, wherein the second connecting member comprises a connection column, the first connecting member is provided with a communication hole, and the connection column penetrates the communication hole and is electrically connected to the second electrode.

3. The atomization core module of claim 2, wherein the first connecting member comprises a first body portion, a surface of an end of the first body portion is provided with a mounting groove, and the mounting groove forms the mounting cavity, and
wherein an atomization surface of the heating unit is disposed toward a bottom surface of the mounting groove.

4. The atomization core module of claim 3, wherein the first electrode is in contact with the bottom surface of the mounting groove so as to electrically connect the first electrode to the first connecting member.

5. The atomization core module of claim 3, wherein a bottom wall of the mounting groove is provided with a first groove, an inner surface of the first groove has a protrusion, and the protrusion extends in a depth direction of the first groove, and
wherein an end surface of the protrusion that is close to the mounting groove is electrically connected to the first electrode.

6. The atomization core module of claim 3, wherein a bottom wall of the mounting groove is provided with a first groove, and an atomization cavity is formed between the atomization surface of the heating unit and an inner surface of the first groove.

7. The atomization core module of claim 6, wherein the first connecting member comprises a first extending portion connected to the first body portion, the first extending portion is provided with a first through hole in communication with the first groove, and the first through hole is configured to communicate the atomization cavity with outside air.

8. The atomization core module of claim 7, wherein an outer diameter of the first extending portion is less than an outer diameter of the first body portion, and
wherein the bottom wall of the first groove is provided with the communication hole.

9. The atomization core module of claim 8, wherein the second connecting member comprises a second body portion and a second extending portion that are connected to each other,
wherein a surface of an end of the second body portion is provided with a second groove, the first body portion being disposed in the second groove,
wherein the second extending portion is provided with a second through hole in communication with the second groove, the first extending portion being disposed in the second through hole, and
wherein the connection column is disposed on a bottom surface of the second groove.

10. The atomization core module of claim 9, wherein an outer diameter of the second extending portion is less than an outer diameter of the second body portion.

11. The atomization core module of claim 9, wherein an outer surface of the second extending portion is provided with a thread so as to connect the atomization core module to a main unit.

12. The atomization core module of claim 9, wherein the insulation member comprises:
a hollow insulation tube disposed between the first extending portion and the second extending portion; and
an annular flange connected to an outer surface of an end of the hollow insulation tube and disposed between the first body portion and a bottom wall of the second groove.

13. The atomization core module of claim 1, wherein the heating assembly comprises a seal member configured to seal a periphery of the heating unit, and
wherein the heating unit comprises a porous liquid guide member and a heating element, the porous liquid guide member comprising a liquid absorbing surface and an atomization surface, the heating element being disposed on the atomization surface.

14. An atomizer, comprising:
an atomization tube comprising a first end and a second end that are opposite each other;
the atomization core module of claim 1 disposed at the first end of the atomization tube and plugging the first end of the atomization tube; and
a suction nozzle component disposed at the second end of the atomization tube and forming a first channel,
wherein the atomization tube, the atomization core module, and the suction nozzle component cooperate to form a liquid storage cavity configured to store an aerosol-generating substrate,
wherein the heating unit of the atomization core module is configured to atomize the aerosol-generating substrate to generate an aerosol, and
wherein the first channel is configured to output the aerosol.

15. The atomizer of claim 14, wherein the first connecting member comprises the first body portion, a surface of the end of the first body portion is provided with the mounting groove, and the mounting groove forms the mounting cavity,
wherein a bottom wall of the mounting groove is provided with the first groove, the atomization cavity being formed between the atomization surface of the heating unit and an inner surface of the first groove, and
wherein the atomization tube forms a second channel that communicates the atomization cavity with the first channel.

16. The atomizer of claim 14, wherein an end portion of the heating assembly that is close to the suction nozzle component is provided with a liquid outlet, the liquid outlet being configured to enable the liquid storage cavity to be in fluid communication with the heating assembly.

17. The atomizer of claim 16, wherein the heating assembly comprises a seal member configured to seal a periphery of the heating unit, and
wherein the seal member comprises at least one notch, the at least one notch forming the liquid outlet.

18. An electronic atomization apparatus, comprising:
the atomizer of claim 14, the atomizer being configured to store and atomize an aerosol-generating substrate; and
a main unit configured to supply power to the atomizer and control the atomizer to atomize the aerosol-generating substrate.
